(19) United States
(12) Patent Application Publication
(10) Pub. No.: US 2022/0280273 A1
(43) Pub. Date: Sep. 8, 2022

(54) INFLATABLE MEDICAL IMPLANT HAVING A PRESSURE CALIBRATION SYSTEM

(71) Applicant: Boston Scientific Scimed, Inc., Maple Grove, MN (US)

(72) Inventors: Noel Smith, Windgap (IE); John Gildea, Kilcock (IE); Brian P. Watschke, Minneapolis, MN (US)

(21) Appl. No.: 17/653,225

(22) Filed: Mar. 2, 2022

Related U.S. Application Data
(60) Provisional application No. 63/200,370, filed on Mar. 3, 2021.

(51) Int. Cl. A61F 2/00

(52) U.S. Cl. CPC A61F 2/004

(57) ABSTRACT

According to an aspect, an apparatus includes a bodily implant configured to be implanted into a body of a patient. The implant having an inflatable member, a sensor, and a calibration module. The inflatable member is configured to be disposed proximate a portion of the body of the patient. The sensor is operatively coupled to the inflatable member and is configured to detect a fluidic pressure within the inflatable member. The calibration module calibration module is configured to receive pressure data from the sensor and determine when the inflatable member is placing a pressure on the portion of the body of the patient.

### CROSS-REFERENCE TO RELATED APPLICATION

This application claims priority to U.S. Provisional Patent Application No. 63/200,370, filed on Mar. 3, 2021, entitled “INFLATABLE MEDICAL IMPLANT HAVING A PRESSURE CALIBRATION SYSTEM”, the disclosure of which is incorporated by reference herein in its entirety.

### TECHNICAL FIELD

This disclosure relates generally to a medical device having an inflatable member and a pressure calibration system.

### BACKGROUND

In some medical devices, an inflatable member or portion is used to apply pressure to a portion of the body. For example, in some medical devices, an inflatable member or portion is used to apply pressure to a urethra of a patient. In some such medical devices, pressure can be applied to the urethra of the patient to help resolve continence issues. In some cases, the medical device may include a pump, such as an electric pump, to inflate or deflate the inflatable member. In some cases, it can be important to not overinflate or over deflate the inflatable member so as to conserve battery power and/or have an otherwise efficient system.

Accordingly, there is a need for a medical device that may be inflated within the body of the patient and includes a calibration system.

### SUMMARY

According to an aspect, an apparatus includes a bodily implant configured to be implanted into a body of a patient. The implant having an inflatable member, a sensor, and a calibration module. The inflatable member is configured to be disposed proximate a portion of the body of the patient. The sensor is operatively coupled to the inflatable member and is configured to detect a fluidic pressure within the inflatable member. The calibration module calibration module is configured to receive pressure data from the sensor and determine when the inflatable member is placing a pressure on the portion of the body of the patient.

In some embodiments, the inflatable member is configured to be disposed in an inflated configuration and a deflated configuration. In some embodiments, the inflatable member is configured to be disposed in an inflated configuration and a deflated configuration, the inflatable member being configured to place a first pressure on the portion of the body of the patient when the inflatable member is in its inflated configuration and a second pressure on the portion of the body of the patient when the inflatable member is in its deflated configuration. In some embodiments, the inflatable member is configured to be disposed in an inflated configuration and a deflated configuration, the inflatable member being configured to place a first pressure on the portion of the body of the patient when the inflatable member is in its inflated configuration and a second pressure on the portion of the body of the patient when the inflatable member is in its deflated configuration, the first pressure being greater than the second pressure.

In some embodiments, the bodily implant includes a pump, the pump being operatively coupled to the inflatable member and configured to pump a fluid out of the inflatable member. In some embodiments, the bodily implant includes a pump, the pump being operatively coupled to the inflatable member and configured to pump a fluid into the inflatable member. In some embodiments, the bodily implant includes a first pump and second pump.

In some embodiments, the bodily implant includes an electric pump. In some embodiments, the bodily implant includes a first electric pump and a second electric pump.

In some embodiments, the bodily implant includes a reservoir configured to hold fluid.

In some embodiments, the calibration module includes an evaluation module, the evaluation module being configured to evaluate pressure data. In some embodiments, the calibration module includes a smoothing module, the smoothing module being configured to smooth pressure data received from the sensor.

In some embodiment, the inflatable member is configured to be disposed proximate a urethra of a patient. In some embodiments, the inflatable member is configured to be disposed in a circular configuration. In some embodiments, the inflatable member is configured to be disposed in a circular configuration and is configured to surround a urethra of a patient.

According to an aspect, an apparatus includes a bodily implant configured to be implanted into a body of a patient, the implant including an inflatable member, a reservoir, a first electrical pump, a second electrical pump, a sensor, and a calibration module, the inflatable member being configured to be disposed proximate a portion of the body of the patient, the first electrical pump being configured to pump fluid from the inflatable member to the reservoir, the second electrical pump being configured to pump fluid from the reservoir to the inflatable member, the sensor is operatively coupled to the inflatable member and is configured to detect a fluidic pressure within the inflatable member, and the calibration module calibration module is configured to receive pressure data from the sensor and determine when the inflatable member is placing a pressure on the portion of the body of the patient.

In some embodiments, the inflatable member is configured to be disposed proximate a urethra of a patient. In some embodiments, the inflatable member is configured to be disposed in a circular configuration and is configured to surround a urethra of a patient.

According to an aspect, a method includes deflating an inflatable member that is disposed within a body of a patient; sensing the pressure applied by the inflatable member to a portion of the body of the patient; and determining when the inflatable member is no longer applying a pressure to the portion of the body of the patient.

In some embodiments, the method includes smoothing pressure data received from a pressure sensor.

### BRIEF DESCRIPTION OF THE DRAWINGS

FIG. 1 schematically illustrates an apparatus according to an embodiment of the invention.

FIG. 2 is a schematically illustrates an apparatus according to another embodiment.

FIGS. 3-6 are graphs illustrating pressure over time of an apparatus according to an embodiment.

FIG. 7 is a flow chart of a method according to an embodiment.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The bodily implant disclosed herein are configured to be disposed within a body of a patient. In some embodiments, the bodily implant includes an inflatable member or inflation member. In some embodiments, the inflatable member is configured to be inflated to place pressure on a portion of the body of the patient. In some embodiments, the bodily implant may be placed within a pelvic region of a patient. In some embodiments, the bodily implant is an artificial urinary sphincter and the inflatable member is configured to place pressure on a urethra of a patient. In other embodiments, the implant may be another type of implant. In other embodiments, the bodily implant is configured to be placed in a different region of the body of the patient and is configured to place pressure on a different portion of the body of the patient.

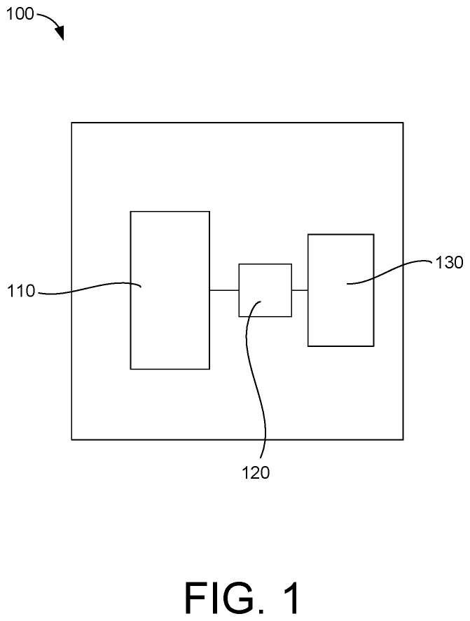

FIG. 1 illustrates an apparatus 100 according to an embodiment of the invention. In the illustrated embodiment, the apparatus or a bodily implant 100. The bodily implant 100 is configured to be disposed or otherwise placed within a body of a patient. In some embodiments, the bodily implant is configured to be placed within a pelvic region of a patient. For example, in some embodiments, the bodily implant is a configured to be placed within a pelvic region of a patient and is configured to address or help treat continence issues of the patient, such as urinary incontinence or fecal incontinence.

In the illustrated embodiment, the bodily implant 100 includes an inflatable or inflation member 110, a sensor 120, and a calibration module 130. The inflatable member 110 is configured to be placed in an inflated configuration and a deflated configuration. In some embodiments, the inflatable member 110 is configured to place pressure on a portion of the body of the patient when the inflatable member 110 is in its inflated configuration. For example, in some embodiments, the inflatable member 110 is configured to be disposed proximate a urethra of a patient and is configured to serve as an artificial sphincter. In such an embodiment, the inflatable member 110 applies a pressure to the urethra when the inflatable member 110 is in its inflated configuration and does not apply a pressure (or applies less of a pressure) when the inflatable member 110 is in is deflated configuration.

In some embodiments, the inflatable member 110 is formed of a material that is configured to expand. In some embodiments, the inflatable member 110 is a balloon or other inflatable type device. In some embodiments, the inflatable member 110 is or forms a loop or circle and is configured to surround a portion of the body of the patient, such as a urethra of a patient.

The sensor 120 is operatively coupled to the inflatable member 110. The sensor 120 is configured to sense or detect a pressure within the inflatable member 110.

The calibration module 130 is operatively coupled to the sensor 120. The calibration module 130 is configured to receive pressure data of the inflatable member 110 from the sensor 120. In some embodiments, the calibration module 130 is configured to determine when the inflatable member 110 is applying pressure to the body of the patient. In some embodiments, the calibration module 130 is configured to determine when the inflatable member 110 is not applying pressure to the body of the patient. In some embodiments, the calibration module 130 is configured to determine when the inflatable member 110 is applying pressure to the body of the patient and when it is not applying pressure to the body of the patient.

In some embodiments, the calibration module 130 is configured to determine the atmospheric pressure of the location of the patient. For example, the calibration module 130 is configured to determine if the local atmospheric pressure is greater than normal (patient is diving in the ocean) or if the local atmospheric pressure is less than normal (patient is hiking a tall mountain).

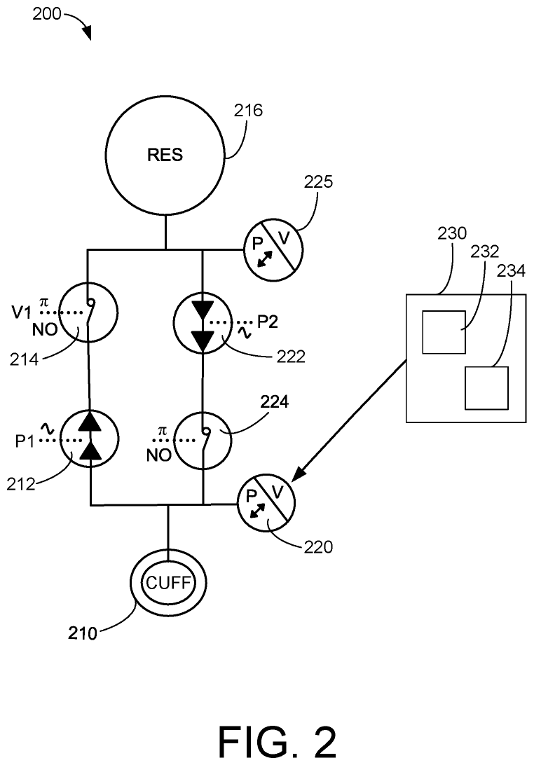

FIG. 2 schematically illustrates a bodily implant 200 according to an embodiment. The bodily implant 200 is configured to be disposed within a body of a patient. In the illustrated embodiment, the bodily implant 200 includes an inflatable or inflation member 210. The inflatable member 210 is configured to be placed in an inflated configuration and a deflated configuration. In some embodiments, the inflatable member 210 is configured to place pressure on a portion of the body of the patient when the inflatable member 210 is in its inflated configuration. In the illustrated embodiment, the inflatable member 210 is a cuff that is configured to surround a urethra of a patient. The inflatable member 210 applies a pressure to the urethra when the inflatable member 210 is in its inflated configuration and does not apply a pressure (or applies less of a pressure) when the inflatable member 210 is in is deflated configuration.

In some embodiments, the inflatable member 210 is formed of a material that is configured to expand. In some embodiments, the inflatable member 210 is a balloon or other inflatable type device.

The bodily implant 200 includes a first pump 212, a first valve 214 and a reservoir 216. The inflatable member 210 is operatively coupled to the first pump 212. For example, a tubular member, such as a kink-resistant tubular member, may be coupled to and extend from the inflatable member 210 to the first pump 212. The first pump 212 is configured to pump fluid out of the inflatable member 210. In the illustrated embodiment, the first pump 212 is configured to pump fluid out of the inflatable member 210 and towards or into the reservoir 216. In some embodiments, the first pump 212 is an electric pump or a pump that operates on an electrical power source.

The first pump 212 is operatively coupled to the first valve 214. For example, a tubular member, such as a kink-resistant tubular member, may be coupled to and extend from the first pump 212 to the first valve 214. The first valve 214 is configured to allow fluid to pass in the direction towards the reservoir 216.

The first valve 214 is operatively coupled to the reservoir 216. A tubular member, such as a kink-resistant tubular member, extends between and couples the first valve 214 to the reservoir.

The reservoir 216 is configured to hold fluid. The reservoir 216 may be a pressure-regulating inflation balloon or element. The reservoir 216 may be constructed of polymer material that is capable of elastic deformation to reduce fluid volume within the fluid reservoir 216 and push fluid out of the fluid reservoir 216. In some embodiments, the reservoir 216 is made from an elastic material and is configured to expand when fluid is disposed in the reservoir 216. In some examples, the fluid reservoir 216 is implanted into the abdominal space.

The bodily implant 200 also includes a second pump 222 and a second valve 224. The reservoir 216 is operatively coupled to the second pump 222. For example, a tubular member, such as a kink-resistant tubular member, may be coupled to and extend from the reservoir 216 to the second pump 222. The second pump 222 is configured to pump fluid into the inflatable member 210. In the illustrated embodiment, the second pump 222 is configured to pump fluid out of the reservoir 216 and towards or into the inflatable member 210. In some embodiments, the second pump 222 is an electric pump or a pump that operates on an electrical power source.

The second pump 222 is operatively coupled to the second valve 224. For example, a tubular member, such as a kink-resistant tubular member, may be coupled to and extend from the second pump 222 to the second valve 224. The second valve 224 is configured to allow fluid to pass in the direction towards the inflatable member 210.

The second valve 224 is operatively coupled to the inflatable member 210. A tubular member, such as a kink-resistant tubular member, extends between and couples the second valve 224 to the inflatable member 210.

The bodily implant 200 also includes a first sensor 220 and a second sensor 225. The first sensor 220 is operatively coupled to the inflatable member 210 and is configured to sense or detect the fluidic pressure within the inflatable member 210. The second sensor 225 is operatively coupled to the reservoir 216 and is configured to sense or detect the fluidic pressure within the reservoir 216.

The bodily implant 200 also includes a calibration module 230. The calibration module 230 is operatively coupled to the first sensor 220. The calibration module 230 is configured to receive pressure data of the inflatable member 210 from the sensor 220. In some embodiments, the calibration module 230 is configured to determine when the inflatable member 210 is applying pressure to the body of the patient. In some embodiments, the calibration module 230 is configured to determine when the inflatable member 210 is not applying pressure to the body of the patient. In some embodiments, the calibration module 230 is configured to determine when the inflatable member 210 is applying pressure to the body of the patient and when it is not applying pressure to the body of the patient.

In some embodiments, the calibration module 230 is configured to determine the atmospheric pressure of the location of the patient. For example, the calibration module 230 is configured to determine if the local atmospheric pressure is greater than normal (patient is diving in the ocean) or if the local atmospheric pressure is less than normal (patient is hiking a tall mountain).

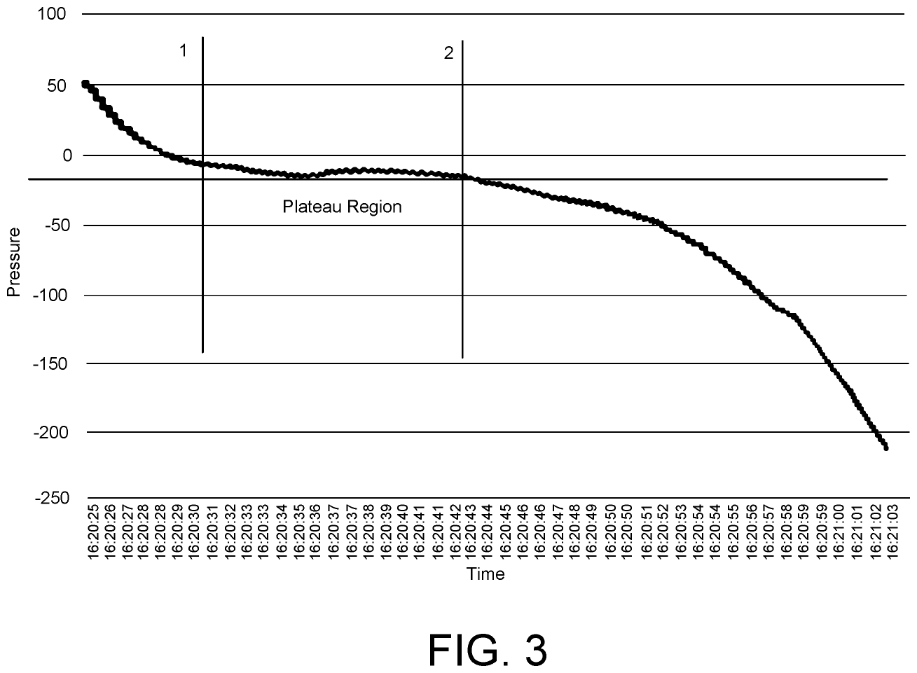

As best illustrated in FIG. 3, the pressure applied by the inflatable member 210 levels out for a period of time (the plateau region). In this region, the inflatable member 210 is no longer applying pressure to the portion of the body of the patient. Accordingly, the calibration module 230 can determine when the pump 212 can cease pumping fluid from the inflatable member 210. Specifically, in some embodiment, the pump 212 can cease pumping fluid from the inflatable member 210 at the beginning of the plateau region so as to conserve power resources.

In the illustrated embodiment, the calibration module 230 includes an evaluation module 232 and a smoothing module 234. The evaluation module 232 is configured to receive and evaluate pressure data received from the first sensor 220. The smoothing module 234 is configured to smooth the pressure date received from the first sensor 220.

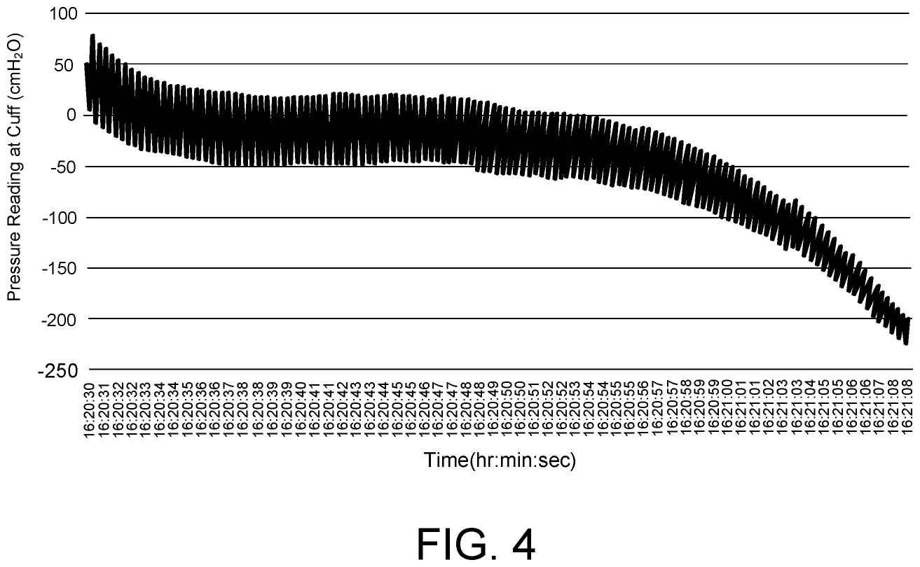

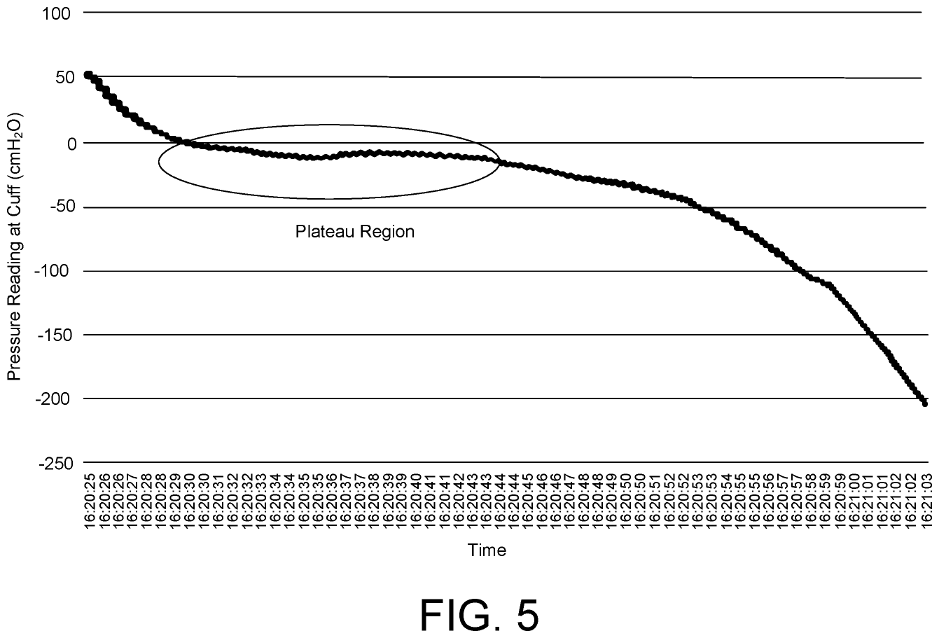

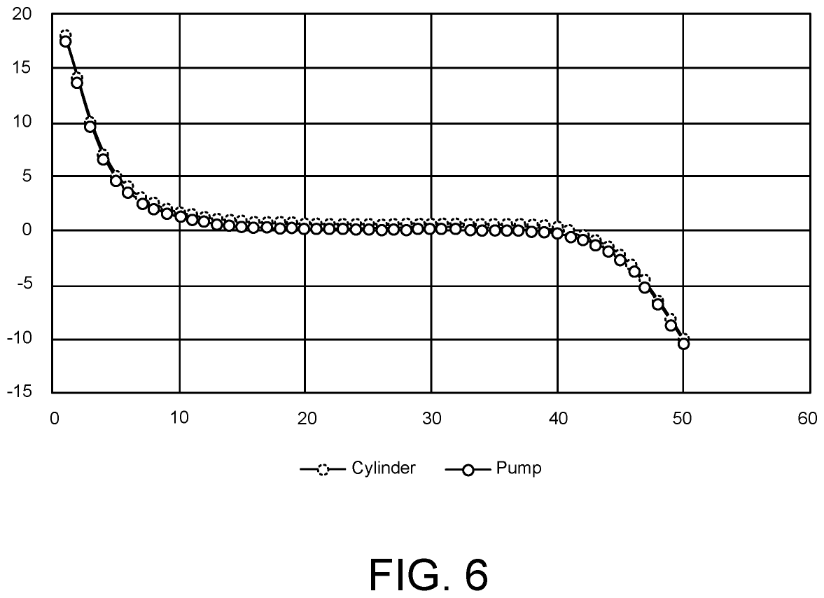

As best illustrated in FIG. 4, in some embodiments, the pressure data received from the first sensor 220 oscillates. In some cases, the oscillation may make the plateau region difficult to identify. Accordingly, as illustrated in FIG. 5, the oscillating data may be smoothed to more easily and more accurately identify the plateau region. FIG. 6 also illustrates pressure data over time.

The smoothing module 234 may use any number of methods for smoothing the oscillating data. For example, in some embodiments, the smoothing module uses a standard deviation method to smooth the data. In this embodiment, the standard deviation of a subset of the data points will be the smallest at the plateau region. In another embodiment, the smoothing module uses a subtraction method to generate a smoothed curve. In such an embodiment, the value of the previous point is subtracted from the value of the current point to smooth the curve. In yet another embodiment, the inflection point or points of the oscillating curve may be identified to identify the plateau region.

In some embodiments, when the inflatable member 210 is being inflated, there may be a higher pressure on at the reservoir 216 than at the inflatable member 210. In such cases, passive filling of the inflatable member 210 may be used. For example, in such cases, the second valve 224 may be opened to allow fluid to flow from the reservoir 216 to the inflatable member 210 without having to operate the second pump 234.

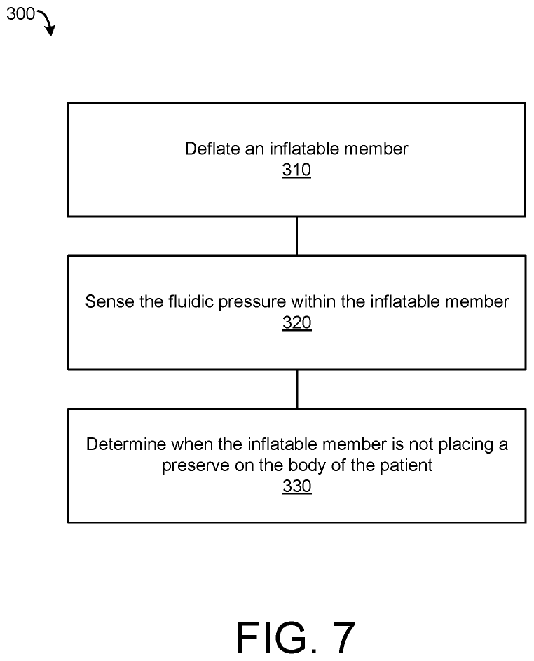

FIG. 7 is a flow chart of a method 300 according to an embodiment of the invention. In the illustrated embodiment, the method 300 includes at 310 deflating an inflatable member that is disposed within a body of the patient. At 320, the fluid pressure of the inflatable member is sensed or detected. At 330, it is determined when the inflatable member is no long placing a pressure on the body of the patient. For example, it can be determined when an inflatable member disposed adjacent a urethra of a patient is no longer placing a pressure on the urethra of the patient.

Various implementations of the systems, modules, and other units described herein, and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. Various implementations of the systems and techniques described here can be realized as and/or generally be referred to herein as a circuit, a module, a block, or a system that can combine software and hardware aspects. For example, a module may include the functions/acts/computer program instructions executing on a processor (e.g., a processor formed on a silicon substrate, a GaAs substrate, and the like) or some other programmable data processing apparatus.

Some of the above example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed above, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a storage medium. A processor(s) may perform the necessary tasks.

Specific structural and functional details disclosed herein are merely representative for purposes of describing example embodiments. Example embodiments, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term and/or includes any and all combinations of one or more of the associated listed items.

The terminology used herein is for the purpose of describing particular embodiments only and is not intended to be limiting of example embodiments. As used herein, the singular forms “a”, “an”, and “the” are intended to include the plural forms as well, unless the context clearly indicates otherwise. It will be further understood that the terms comprises, comprising, includes and/or including, when used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the above example embodiments and corresponding detailed description are presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the above illustrative embodiments, reference to acts and symbolic representations of operations (e.g., in the form of flowcharts) that may be implemented as program modules or functional processes include routines, programs, objects, components, data structures, etc., that perform particular tasks or implement particular abstract data types and may be described and/or implemented using existing hardware at existing structural elements. Such existing hardware may include one or more Central Processing Units (CPUs), digital signal processors (DSPs), application-specific-integrated-circuits, field programmable gate arrays (FPGAs) computers or the like.

It should be borne in mind, however, that all of these and similar terms are to be associated with the appropriate physical quantities and are merely convenient labels applied to these quantities. Unless specifically stated otherwise, or as is apparent from the discussion, terms such as processing or computing or calculating or determining of displaying or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

Note also that the software implemented aspects of the example embodiments are typically encoded on some form of non-transitory program storage medium or implemented over some type of transmission medium. The program storage medium may be magnetic (e.g., a floppy disk or a hard drive) or optical (e.g., a compact disk read only memory, or CD ROM), and may be read only or random access. Similarly, the transmission medium may be twisted wire pairs, coaxial cable, optical fiber, or some other suitable transmission medium known to the art. The example embodiments not limited by these aspects of any given implementation.

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to employ the implementations in virtually any appropriately detailed structure. Further, the terms and phrases used herein are not intended to be limiting, but to provide an understandable description of the present disclosure.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
a bodily implant configured to be implanted into a body of a patient, the implant including an inflatable member, a sensor, and a calibration module,
the inflatable member being configured to be disposed proximate a portion of the body of the patient,
the sensor is operatively coupled to the inflatable member and is configured to detect a fluidic pressure within the inflatable member, and
the calibration module calibration module is configured to receive pressure data from the sensor and determine when the inflatable member is placing a pressure on the portion of the body of the patient.

2. The apparatus of claim 1, wherein the inflatable member is configured to be disposed in an inflated configuration and a deflated configuration.

3. The apparatus of claim 1, wherein the inflatable member is configured to be disposed in an inflated configuration and a deflated configuration, the inflatable member being configured to place a first pressure on the portion of the body of the patient when the inflatable member is in its inflated configuration and a second pressure on the portion of the body of the patient when the inflatable member is in its deflated configuration.

4. The apparatus of claim 1, wherein the inflatable member is configured to be disposed in an inflated configuration and a deflated configuration, the inflatable member being configured to place a first pressure on the portion of the body of the patient when the inflatable member is in its inflated configuration and a second pressure on the portion of the body of the patient when the inflatable member is in its deflated configuration, the first pressure being greater than the second pressure.

5. The apparatus of claim 1, wherein the bodily implant includes a pump, the pump being operatively coupled to the inflatable member and configured to pump a fluid out of the inflatable member.

6. The apparatus of claim 1, wherein the bodily implant includes a pump, the pump being operatively coupled to the inflatable member and configured to pump a fluid into the inflatable member.

7. The apparatus of claim 1, wherein the bodily implant includes a first pump and second pump.

8. The apparatus of claim 1, wherein the bodily implant includes an electric pump.

9. The apparatus of claim 1, wherein the bodily implant includes a first electric pump and a second electric pump.

10. The apparatus of claim 1, wherein the bodily implant includes a reservoir configured to hold fluid.

11. The apparatus of claim 1, wherein the calibration module includes an evaluation module, the evaluation module being configured to evaluate pressure data.

12. The apparatus of claim 1, wherein the calibration module includes a smoothing module, the smoothing module being configured to smooth pressure data received from the sensor.

13. The apparatus of claim 1, wherein the inflatable member is configured to be disposed proximate a urethra of a patient.

14. The apparatus of claim 1, wherein the inflatable member is configured to be disposed in a circular configuration.

15. The apparatus of claim 1, wherein the inflatable member is configured to be disposed in a circular configuration and is configured to surround a urethra of a patient.

16. An apparatus, comprising:
a bodily implant configured to be implanted into a body of a patient, the implant including an inflatable member, a reservoir, a first electrical pump, a second electrical pump, a sensor, and a calibration module,
the inflatable member being configured to be disposed proximate a portion of the body of the patient,
the first electrical pump being configured to pump fluid from the inflatable member to the reservoir,
the second electrical pump being configured to pump fluid from the reservoir to the inflatable member,
the sensor is operatively coupled to the inflatable member and is configured to detect a fluidic pressure within the inflatable member, and
the calibration module calibration module is configured to receive pressure data from the sensor and determine when the inflatable member is placing a pressure on the portion of the body of the patient.

17. The apparatus of claim 16, wherein the inflatable member is configured to be disposed proximate a urethra of a patient.

18. The apparatus of claim 16, wherein the inflatable member is configured to be disposed in a circular configuration and is configured to surround a urethra of a patient.

19. A method, comprising:
deflating an inflatable member that is disposed within a body of a patient;
sensing the pressure applied by the inflatable member to a portion of the body of the patient; and
determining when the inflatable member is no longer applying a pressure to the portion of the body of the patient.

20. The method of claim 19, further comprising:
smoothing pressure data received from a pressure sensor.
